(19) United States
(12) Patent Application Publication
(10) Pub. No.: US 2013/0115303 A1
(43) Pub. Date: May 9, 2013

(54) Natural Treatment for Baldness Prevention

(71) Applicant: Eden Variety Products, LLC, Roselle, NJ (US)

(72) Inventor: Wilfredo Ganuza, Roselle, NJ (US)

(73) Assignee: Eden Variety Products, LLC, Roselle, NJ (US)

(21) Appl. No.: 13/667,214

(22) Filed: Nov. 2, 2012

Related U.S. Application Data
(60) Provisional application No. 61/555,707, filed on Nov. 4, 2011.

(51) Int. Cl. A61K 35/64; A61P 17/14

(52) U.S. Cl. 424/537

(57) ABSTRACT

An all natural formulation for the treatment of baldness in men and women is described. Also described is a method of making the formulation and a method of using it.

### CLAIM OF PRIORITY

This application claims the priority of U.S. Ser. No. 61/555,707 filed on Nov. 4, 2011, the contents of which are fully incorporated herein by reference.

### FIELD OF THE INVENTION

The invention relates to a composition useful in treating baldness, in particular to a composition of natural ingredients and a method of using the composition.

### BACKGROUND OF THE INVENTION

The invention relates to a composition, a method of making the composition, and a method of using the composition to slow hair loss and to aid in hair re-growth. Anecdotal evidence indicates that the invention is effective in both men and women.

Alopecia, or hair loss, is a common cosmetic problem that can also cause serious negative psychological effects. Hair growth is cyclical, occurring in three stages: anagen, the active growth phase; catagen, the degenerative phase; and telogen, the resting phase. After telogen, the old hair fiber is shed and a new hair is generated as part of the repeating cycle. Alopecia, or hair loss, occurs in both men and women, and is attributed to numerous causes including aging, hormone levels, stress, and chemotherapy. In these circumstances, more and more hair follicles remain in the telogen stage, resulting in a gradual decrease of the hair fiber length and diameter, finally reaching a stage of partial or complete baldness.

Various types of hair loss are known, including alopecia areata, androgenetic alopecia, anagen effluvium, self-induced hair loss, telogen effluvium, and scarring alopecia. Alopecia areata, thought to be an auto-immune disorder, begins with hair loss in a rounded patch on the scalp. Alopecia areata includes mild patchy hair loss on the scalp, as well the loss of all scalp hair, known as alopecia totalis, and the loss of all scalp and body hair, known as alopecia universalis. Androgenetic alopecia, including male and female pattern baldness, is thought to be caused by a combination of genetic predisposition, aging, and androgen hormone levels.

Androgenetic alopecia is associated with increased androgen stimulation, which adversely affects the hair follicles. Increased androgen stimulation can be produced, among other mechanisms, by elevated levels of 5-alpha-reductase, an enzyme that converts testosterone to dihydrotestosterone. Anagen effluvium is hair loss due to chemicals or radiation, such as chemotherapy or radiation treatment for cancer. Self-induced hair loss includes hair loss caused by conscious or unconscious self-inflicted damage to the hair. Two common types of self-induced hair loss are trichotillomania, or hair loss that results when someone continually pulls or plucks out his own hair, and traction alopecia, which is caused by hairstyles such as ponytails or braids that continually pull at the hair. Telogen effluvium is stress-related hair loss caused by events such as, for example, surgery, child birth, or pregnancy termination. Further causes of telogen effluvium include the use of oral contraceptives or other prescription drugs, thyroid abnormality, diabetes, lupus, and emotional stress. Scarring alopecia includes hair loss caused by infection and inflammation of the hair follicles, and hair loss caused by burns or other trauma.

Because hair loss is a widespread problem that is considered cosmetically unappealing and often causes emotional distress, there is great demand for alopecia treatments. Many compositions have been tested for their ability to stimulate hair growth, for example, by promoting or prolonging anagen. Examples of such compositions include potassium channel openers, such as minoxidil (Rogaine®, Pharmacia Corp.) and diazoxide; 5-alpha-reductase inhibitors, such as finasteride (Propecia®, Merck & Co.); and the immunosuppressant cyclosporin A. However, known treatments for stimulating hair growth exhibit limited effectiveness and cause unwanted side effects. For example, among other undesirable side effects, potassium channel openers cause cardiovascular effects, finasteride is unsafe for women who are pregnant or may become pregnant, and cyclosporin A suppresses the immune system. Further, even when applied topically to areas in which hair growth is desired, known treatments for alopecia often cause hair growth in undesired areas of the body, such as facial hair on women. Such disadvantages of known compositions for treating alopecia lead many individuals experiencing hair loss to rely on wigs and toupees. Other individuals seek hair transplant surgery, which is expensive, is not fully effective, and sometimes is not possible, for example, for chemotherapy patients. Accordingly, there is a need for new agents for treating alopecia that are safe and effective and stimulate hair growth only in desired areas.

Various treatments are known in the art, but fail to address all of the problems solved by the invention described herein.

### SUMMARY OF THE INVENTION

The present invention is a composition comprising 5-90% by weight Aloe vera slurry liquid portion and 1-90% by weight honey. The composition may also comprise any or all of the extracts from the parent constituents of chamomile, rosemary, horse tail, and sage. The composition is applied to the skin in a place in which a person wants to grow hair. It has been shown effective in non-clinical testing to re-grow hair and to stop hair loss when used as directed.

Therefore, the present invention succeeds in conferring the following, and others not mentioned, desirable and useful benefits and objectives.

It is an object of the present invention to provide a treatment for preventing hair loss.

It is another object of the present invention to provide a treatment for re-growing hair.

Yet another object of the present invention is to provide a natural treatment for treating hair loss.

It is an object of the present invention to prevent dandruff.

It is another object of the present invention to provide a treatment for animals.

It is another object of the present invention to provide a shampoo.

It is another object of the present invention to provide a treatment for acne.

### DESCRIPTION OF THE PREFERRED EMBODIMENTS

Reference will now be made in detail to embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

The present invention is a composition of components. The components are, with the exception of honey, extracts of their parent constituents. The extraction process will be described in detail with regard to the examples that are cited after the description of the parent constituents below. The parent constituents may be ground up, used whole, crushed, or prepared in any other way for the extraction process. The entire constituent plant may be used, or a part or parts of the constituent plant may be used. For example, only the leaves of the sage plant may be used, only the stems may be used, a combination of leaves and stems may be used, or the entire plant may be used. The components may be extracted from parent constituents prepared in any way, including but not limited to, fresh (no special preparation) or dried.

Aloe vera is a species of succulent plant in the genus Aloe. Aloe vera is about 95% water. The rest contains active ingredients including essential oil, amino acids, minerals, vitamins, enzymes and glycoproteins. Some positive effects of treatment with aloe vera are thought to be due to the presence of compounds such as polysaccharides, mannans, anthraquinones, and lectins.

Other compounds, such as tannins, polysaccharides, organic acids, enzymes, vitamins, and steroids, have been identified. Aloe vera contains bradykininase, which relieves pain and decreases swelling and redness. Magnesium lactate may contribute to the antipruritic effect of aloe by blocking histamine production. An antiprostaglandin that reduces inflammation has also been isolated. Anthraquinones are local irritants in the GI tract but have been used in treating certain skin diseases, such as psoriasis.

Aloe vera has been used externally to treat various skin conditions, such as cuts, burns, and eczema, as well as sunburn, dermatitis, insect stings, poison ivy, abrasions, and other dermatological conditions. It is alleged that sap from Aloe vera eases pain and reduces inflammation. It is held that components such as several glycoproteins and salicylates are anti-inflammatory, and there are substances that stimulate growth of skin and connective tissue, as well as various vitamins and minerals (vitamin C, vitamin E, and zinc) and antifungal and antibacterial components (Longe 2005). Aloe vera's activity is also attributed to moisturizing and pain relieving properties (Longe 2005).

Anecdotal evidence also has indicated that Aloe vera can help in keeping the skin flexible, and it has been used in the control of acne and eczema. The healing power is believed to be due to Aloe vera's action in increasing the availability of oxygen to the skin, and by increasing the strength of skin tissue. The herb has a moisturizing effect on the skin and is a common therapy for sunburn and skin irritation.

Honey is a naturally occurring food made by bees using nectar from flowers. Honey is primarily a saturated mixture of two monosaccharides, dextrose and levulose, with a low water activity. When honey is used topically hydrogen peroxide is produced by dilution of the honey with body fluids and/or water. As a result, hydrogen peroxide is released slowly and acts as an antiseptic.

A review in the Cochrane Library suggests honey could reduce the time it takes for a burn to heal—up to four days sooner in some cases. The review included 19 studies with 2,554 participants.

Chamomile is a plant belonging to the sunflower or daisy family, Asteraceae. The common name Chamomile includes more than one species. Although any species may be used, the preferred species is Matricaria. The flowers of chamomile contain 0.4-2% (v/w) essential oil. The pharmacopoeial standard of essential oil contains no less than 4 mg/kg of blue oil. Its main components are (−)-alpha-bisabolol (up to 50%) and chamazulene (1-15%). The chief constituents of Roman chamomile oil are n-butyl angelate and isoamyl angelate. The flower heads have been shown to contain approximately 0.6% bitter sesquiterpene lactones (germacranolides), including nobilin and its derivatives. Other constituents may include (−)-alphabisabolol oxide A, B and C, (−)-alpha-bisabolone oxide A, 1,8-cineole, en-yn-dicycloether, alpha-pinene, amyl and isobutyl alcohols, angelic acid esters, anthemol, anthemic acid, apigenin, choline, coumarins, farnesol, germacranolide, heniarin, inositol, lueteolin, nerolidol, nobilin, patuletin, phenolic and fatty acids, phytosterol, quercetin, scopoletin-7-glucoside, spiroethers (e.g., cis- and trans-en-yn-dicycloether), sesquiterpenes (e.g., anthecotulid), tricontane, cadinene, farnesene, furfural, spanthulenol, tiglic acid esters, flavonoids (e.g., apigenin and luteolin), proazulenes (e.g., matricarin and matricin), and umbelliferone.²⁹ Chamazulene is formed from matricin during steam distillation of the oil. It varies in yield depending on the origin and age of the flowers.

Up to 50% of essential oil of chamomile contains alpha-bisabolol (terpenoid) and is reported to have anti-inflammatory, antibacterial, antimycotic, (anti-fungal) and ulcer-protective properties. The precise mechanism of action remains unclear, but it has been theorized that azulenes activate the pituitary-adrenal system to release cortisone and prevent the discharge of histamine.

Rosemary is a plant belonging to the mint family, Lamiaceae. Its scientific name is Rosmarinus officinalis. The leaves contain 0.5% to 2.5% of volatile oil. The major components of the oil include monoterpene hydrocarbons (alpha and beta-pinene), camphene, limonene, camphor (10% to 20%), borneol, cineole, linalool, and verbinol. Rosemary contains a wide variety of volatile and aromatic components. Flavonoids in the plant include diosmetin, diosmin, genkwanin, luteolin, hispidulin, and apigenin. One analysis reports new flavonoid glucuronides, also found in the leaves. Other terpenoid constituents in rosemary include triterpenes oleanolic and ursolic acids and diterpene carnosol. The concentration of phenolic diterpenes in certain commercial rosemary extracts has been determined by HPLC. Phenols in rosemary include caffeic, chlorogenic, labiatic, neochlorogenic, and rosmarinic acids. Rosemary contains high amounts of salicylates.

Rosemary extracts are commonly found as cosmetic ingredients and a lotion of the plant is said to stimulate hair growth and prevent baldness. The plant has been used in traditional medicine for its astringent, tonic, carminative, antispasmodic, and diaphoretic properties.

Horsetail is a plant of the family Equisetum. In the context of this application the term “Horsetail” means the various species of the family Equisetum, including but not limited to Equisetum arvense, E. palustris, E. sylvaticum and E. pratense. The stems of horsetail contain 5% to 8% of silica and silicic acids. The plant contains about 5% of a saponin called equisetonin, in addition to the flavone glycosides isoquercitrin, equisetrin, and galuteolin. The sterol fraction of E. arvense contains beta-sitosterol, campestrol, isofucosterol, and trace amounts of cholesterol. The alkaloid nicotine is present in minute amounts (less than 1 ppm) but may account for a portion of the pharmacologic activity of the plant. The plant contains more than 15 types of bioflavonoids, as well as manganese, potassium, sulfur, and magnesium. The cytokinin isopentenyladenosine has been identified in fertile fronds.

Horsetail has been traditionally used for a variety of complaints and conditions, including baldness, tuberculosis, kidney and bladder infections, kidney stones, excessive menstrual flow, gastrointestinal bleeding, gout, skin wounds and ulcers, fractures, frostbite, brittle fingernails, fluid retention, and arthritis. Horsetail is a popular herbal remedy for a variety of conditions, but it has found the most use among people who want to take advantage of its silicon content for improving hair, skin and nail quality.

The genus Salvia includes a number of species that are traditionally used as herbs and called “sage”. The scientific name of the common sage is Salvia officinalis In the context of this invention the term “Sage” includes various Salvia species, including but not limited to Salvia officinalis and Salvia lavandulaefolia. The species Salvia officinalis contains 1% to 2.8% essential oil, along with flavones, phenolic acids, phenylpropanoid glycosides (eg, martynoside), triterpenoids, and diterpenes, including phenolic, quinoidal, and rearranged abietane and apianane derivatives. The plant's compounds include salvigenin, lupeol, beta-sitosterol, stigmasterol, physcion, carnosol, rosmadial, rosmanol, epirosmanol, isorosmanol, columbaridione, atuntzensin A, miltirone, carnosic acid, and 12-O-methyl carnosic acid.

Monoturpenes have been identified using gas chromatography and other techniques, with alpha- and beta-thujones accounting for about one-half of the oil's composition. Capillary electrophoresis has been used to identify the polyphenols, while high performance liquid chromatography and nuclear magnetic resonance techniques have been applied to cold water extracts in identifying polysaccharides.

Salvia lavandulaefolia (Spanish sage) and Salvia officinalis have similar compositions except that Salvia officinalis has a much higher concentration of thujone, which is toxic in large doses. Salvia lavandulaefolia also contains variable amounts of camphor, cineol, limonene, camphene, and pinene. Sage oil is often adulterated by the addition of thujone derived from the leaves of Juniperus virginiana (red cedar).

The plant has been used topically as an antiseptic and astringent and to manage excessive sweating. Sage tea has been ingested for the treatment of dysmenorrhea, diarrhea, gastritis, tonsillitis, and sore throat. The dried leaves have been smoked to treat asthma.

Examples of the compositions made from the components are described below:

### Example 1

Example 1 results in a composition that is 5-98% by weight Aloe vera slurry liquid portion and 1-80% by weight honey.

1) Puree entire fresh Aloe vera leaves in a blender on high speed for approximately 3 minutes such that approximately 570 grams of a slurry is obtained.

2) Pass the slurry of 1) through a separator to separate the liquids from the solids to yield approximately 510 grams of liquid portion.

3) Add approximately 60 grams of honey to the liquid portion obtained from 2) to yield approximately 570 grams of the composition.

4) Mix the components of 3) together until well blended.

### Example 2

Example 2 results in a composition that is 5-98% by weight Aloe vera slurry liquid portion, 1-80% by weight honey, and 0.0001-50% by weight chamomile extract, extracted from chamomile, a parent constituent.

1) Puree entire fresh Aloe vera leaves in a blender on high speed for approximately 3 minutes such that approximately 570 grams of a slurry is obtained.

2) Add approximately 1 gram of chamomile to approximately 140 grams of the slurry obtained in 1) and mix well.

3) Add the remaining approximately 430 grams of slurry from 1) through a separator and retain the liquid.

4) Allow the mixture of 2) to mull for approximately 24 hours at 35-40° C. (95-104° F.).

5) Pass the mulled mixture of 4) through a separator to separate the liquid from the solids.

6) Combine the liquids of steps 3) and 5).

7) Add approximately 60 grams of honey to the liquid obtained from 6).

8) Mix the components of 7).

### Example 3

Example 3 results in a composition that is 5-98% by weight Aloe vera slurry liquid portion, 1-80% by weight honey, and 0.0001-50% by weight rosemary extract, extracted from rosemary, a parent constituent.

1) Puree entire fresh Aloe vera leaves in a blender on high speed for approximately 3 minutes such that approximately 570 grams of a slurry is obtained.

2) Add approximately 0.5 grams of rosemary to approximately 140 grams of the slurry obtained in 1) and mix well.

3) Add the remaining approximately 430 grams of slurry from 1) through a separator and retain the liquid.

4) Allow the mixture of 2) to mull for approximately 24 hours at 35-40° C. (95-104° F.).

5) Pass the mulled mixture of 4) through a separator to separate the liquid from the solids.

6) Combine the liquids of steps 3) and 5).

7) Add approximately 60 grams of honey to the liquid obtained from 6).

8) Mix the components of 7).

### Example 4

Example 4 results in a composition that is 5-98% by weight Aloe vera slurry liquid portion, 1-80% by weight honey, and 0.0001-50% by weight horsetail extract, extracted from horsetail, a parent constituent.

1) Puree entire fresh Aloe vera leaves in a blender on high speed for approximately 3 minutes such that approximately 570 grams of a slurry is obtained.

2) Add approximately 1 gram of horsetail to approximately 140 grams of the slurry obtained in 1) and mix well.

3) Add the remaining approximately 430 grams of slurry from 1) through a separator and retain the liquid.

4) Allow the mixture of 2) to mull for approximately 24 hours at 35-40° C. (95-104° F.).

5) Pass the mulled mixture of 4) through a separator to separate the liquid from the solids.

6) Combine the liquids of steps 3) and 5).

7) Add approximately 60 grams of honey to the liquid obtained from 6).

8) Mix the components of 7).

### Example 5

Example 5 results in a composition that is 5-98% by weight Aloe vera slurry liquid portion, 1-98% by weight honey, and 0.0001-50% by weight sage extract, extracted from sage, a parent constituent.

1) Puree entire fresh Aloe vera leaves in a blender on high speed for approximately 3 minutes such that approximately 570 grams of a slurry is obtained.

2) Add approximately 0.5 grams of sage to approximately 140 grams of the slurry obtained in 1) and mix well.

3) Add the remaining approximately 430 grams of slurry from 1) through a separator and retain the liquid.

4) Allow the mixture of 2) to mull for approximately 24 hours at 35-40° C. (95-104° F.).

5) Pass the mulled mixture of 4) through a separator to separate the liquid from the solids.

6) Combine the liquids of steps 3) and 5).

7) Add approximately 60 grams of honey to the liquid obtained from 6).

8) Mix the components of 7).

### Example 6

Example 6 results in a composition that is 5-98% by weight Aloe vera slurry liquid portion, 1-80% by weight honey, 0.0001-50% by weight chamomile extract, 0.0001-50% by weight rosemary extract, 0.0001-50% by weight horse tail extract, and 0.0001-50% by weight sage extract.

Example 6 may also result in a composition that is 5-98% by weight Aloe vera slurry liquid portion, 1-80% by weight honey, 0.0001-5% by weight chamomile extract, 0.0001-5% by weight rosemary extract, 0.0001-5% by weight horse tail extract, and 0.0001-5% by weight sage extract.

Example 6 may also result in a composition that is 50-95% by weight Aloe vera slurry liquid portion, 1-50% by weight honey, 0.0001-5% by weight chamomile extract, 0.0001-5% by weight rosemary extract, 0.0001-5% by weight horse tail extract, and 0.0001-5% by weight sage extract.

1) Puree entire fresh Aloe vera leaves in a blender on high speed for approximately 3 minutes such that approximately 570 grams of a slurry is obtained.

2) Divide the slurry from 1) into four portions of approximately 140 grams each.

3) Add 0.1 to 10 grams, preferably approximately 1.0 grams of chamomile to one portion of the slurry from 1).

4) Add 0.1 to 10 grams, preferably approximately 0.5 grams of rosemary to a second portion of the slurry from 1).

5) Add 0.1 to 10 grams, preferably approximately 1 gram of horsetail to a third portion of the slurry from 1).

6) Add 0.1 to 10 grams, preferably approximately 0.5 grams of sage to the fourth portion of the slurry from 1).

7) Allow the slurry mixtures of 3-6 to mull separately for 24 hours at 35-40° C. (95-104° F.).

8) Place the slurry mixtures of 7) in a blender and mix them together.

9) Pass the mixture of 8) through a separator to separate the liquids from the solids to yield approximately 510 grams of liquid.

10) Add 1-90% by weight, preferably approximately 60 grams of honey to the liquid obtained from 9).

10) Mix the components of 11) together until well blended.

As per Claim 2, other examples not detailed here include combining the slurry mixtures of two, three, or all four of the parent constituents in various combinations.

Although example 6 details an order for adding the four parent constituents to the Aloe vera slurry portions, and examples 2-6 detail a specific period for mulling, the parent constituents may be added to their respective slurry portions in any order, and the mulling periods may be any length, preferably from 30 minutes to 30 days, and most preferably approximately 24 hours. The mulling periods for each parent constituent need not be the same, although that is preferred.

As stated earlier, the components with the exception of honey are extracted from their parent constituents. The fresh Aloe vera plant is pureed into a slurry; the slurry extracts the components from their parent constituents through diffusion and/or a solvent effect. As can be seen in examples 2-6 above, the parent constituents are mixed with the Aloe vera slurry and are subsequently allowed to mull for 24 hours after each addition; diffusion occurs, allowing molecules to move from the parent constituents into the Aloe vera slurry. The mulling temperature may be any temperature, but is preferably from 20° C. to 60° C. In the examples above, the mulling occurs at the preferred elevated temperature of 35-40° C. (95-104° F.); this speeds the extraction process, but the mulling could also occur at a lower temperature. In this case, the mulling time may be adjusted to compensate for a lower extraction rate. In the preferred embodiment, the parent constituents are all mulled at approximately the same temperature, but different temperatures or times may be used for different components or parent constituents.

The blender used to make the slurry and to mix the separate slurries may be any kind of blender. It preferably is an electric blender such as but not limited to, blenders for home use such as Waring, Hamilton Beach, or Oster, or industrial or commercial blenders by those manufacturers or others. The blender may also be manual, and may be any type of blending instrument, including but not limited to, a hand mixer, a mortar and pestle, a pastry blender, a cement mixer, an industrial mixer, or others.

The separator used may be any type of device that can separate solids from liquids. It is preferably a mesh sieve with sieve size 0.020 mm to 11.2 mm. The separator may also be, but is not limited to, a centrifuge, a filter, a membrane, a cyclone separator, a pressure driven separator, a vacuum separator, or a funnel.

Although the extraction process described in the examples above is the preferred method for obtaining the extracts, the extracts may be obtained through other methods. For instance, other methods of extraction may include, but are not limited to, using different meld times, different temperatures, using varying methods to grind up the parent constituents, using solvents to aid in extraction, using an agitator to facilitate extraction, or they may be purchased from a supplier who uses his own method of extraction.

A composition made using from any of the examples above is applied to the skin in the following manner. First, a user identifies where he or she wants to grow or retain hair. The spot (or spots) is cleaned, then the composition is applied in a thin layer on the skin. The composition is allowed to dry on the user's skin, and is left on for approximately 8 hours or until the skin is again cleaned. The skin is then cleaned again, and the composition is re-applied as above. This process continues as long as the user desires.

The present invention has been used on humans and dogs both, and has been shown anecdotally to reduce dandruff in both species and to reduce skin itching in dogs. It has also been shown to slow hair loss and to re-grow hair in humans. The invention may be used on other species as well to improve skin health.

Other components that may be used to formulate the base for the compositions described above include, but are not limited to, water, vp/va copolymer (polyvinylpyrrolidone/vinyl acetate copolymer), pvp (polyvinyl pyrrolidone), polysorbate 20, ginseng root extract, sage leaf extract, allatoin, panthenol, acrylates, c10-30 alkyl acrylate crosspolymer, propylene glycol, triethanolamine, tetrasodium edta, tetrasodium pyrophosphate, salicylate, limonene, linalool, phenoxyethanol, and diazolidinyl urea.

Additional components may include, but are not limited to, toronjil (scientific name Melissa Officinalis); fragrance, including but not limited to bubble gum fragance; avocado oil; and lemon grass.

An alternate embodiment is a shampoo using any or all of the same components described above or in subsequent paragraphs. The method of preparing the compositions would be the same or similar, but other compounds may be added to formulate the shampoo. These compounds include, but are not limited to, water, decyl glucoside, coco betaine, lauramide dea, lauryl glucoside, edta, methylchloroisothiazolinone, methylisothiazolone, citric acid, sodium chloride, vitamin A, vitamin D, vitamin C, hemp oil, avocado oil, and coconut oil. Another embodiment is a conditioner using any or all of the same components described above or in subsequent paragraphs. Other components of the conditioner may be, but are not limited to, glycerin, emulsifying wax nf, mineral oil, quanternium-7, pvp (polyvinyl pyrrolidone), glycerilstearate se, stearalkonium chloride, ethoxydiglycol, propylene glycol, butylene glycol, extract of matricaria, extract of nettle, extract of birch sap, extract of arnica, extract of cinchona, extract of birch leaf, potassium sorbate, sodium benzoate, and imidasolidinyl urea.

Vitamin C crystals and debittered brewer's yeast powder may be also be added to any of the compositions described above.

Any and/or all of the components described above may be used to formulate a shampoo or skin wash for dogs or other animals.

The present invention has also been shown through use to improve skin health when used as a face wash, particularly in the reduction of acne. It may also be used as a general body wash. It may be combined with other compounds to make these formulations.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed:

1. A composition comprising:
5-98% by weight Aloe vera slurry liquid portion and 1-80% by weight honey.

2. The composition of claim 1, further comprising one or more components from the group consisting of:
0.0001-50% by weight chamomile extract, extracted from chamomile and being a parent constituent,
0.0001-50% by weight rosemary extract, extracted from rosemary and being a parent constituent,
0.0001-50% by weight horse tail extract, extracted from horse tail and being a parent constituent,
0.0001-50% by weight sage extract, extracted from sage and being a parent constituent,
and mixtures thereof.

3. The composition of claim 1, further comprising:
0.0001-50% by weight chamomile extract extracted from chamomile,
0.0001-50% by weight rosemary extract extracted from rosemary,
0.0001-50% by weight horse tail extract, extracted from horsetail, and
0.0001-50% by weight sage extract extracted from sage leaves.

4. The composition of claim 1, further comprising:
0.0001-5% by weight chamomile extract extracted from chamomile,
0.0001-5% by weight rosemary extract extracted from rosemary,
0.0001-5% by weight horse tail extract, extracted from horsetail, and 0.0001-5% by weight sage extract extracted from sage leaves.

5. The composition of claim 4, further comprising 50-95% by weight Aloe vera slurry liquid portion and 1-50% by weight honey.

6. A method of making the composition of claim 1, comprising:
a) pureeing fresh Aloe vera leaves to yield a slurry;
b) passing the slurry of a) through a separator to separate the liquids from the solids;
c) adding 1-80% by weight honey to the liquid obtained from b);
d) mixing the components of c), such that the resulting mixture is 5-98% Aloe vera and 1-80% honey.

7. A method for making the composition of claim 2, comprising;
a) pureeing fresh Aloe vera leaves to yield a slurry;
b) mixing 0.1 to 10 grams of one or more parent constituents into separate 10 to 1000 gram portions of slurry and subsequently waiting a period of approximately 24 hours;
c) mixing the separate slurry mixtures of b) to form one slurry mixture;
d) passing the slurry mixture of c) through a separator to separate the liquids from the solids;
e) adding 1-90% by weight of honey to the liquid obtained from d);
f) mixing the components of e).

8. A method for making the composition of claim 3, comprising;
a) pureeing fresh Aloe vera leaves to yield a slurry;
b) mixing 0.1 to 10 grams of each parent constituent into a separate portion of slurry that is approximately 10 to 1000 grams, waiting 24 hours after each addition;
c) mixing the separate slurry mixtures to form one slurry mixture of approximately 40 to 4000 grams;
d) passing the slurry mixture of c) through a separator to separate the liquids from the solids;
e) adding 1-90% by weight of honey to the liquid obtained from d);
f) mixing the components of e).

9. A method for making the composition of claim 4, comprising;
a) pureeing fresh Aloe vera leaves to yield a slurry;
b) mixing 0.1 to 5 grams of each parent constituent into a separate portion of slurry that is approximately 10 to 1000 grams, waiting 24 hours after each addition;
c) mixing the separate slurry mixtures to form one slurry mixture of approximately 40 to 4000 grams;
d) passing the slurry mixture of c) through a separator to separate the liquids from the solids;
e) adding 1-90% by weight of honey to the liquid obtained from d);
f) mixing the components of e).

10. A method for making the composition of claim 5, comprising;
a) pureeing fresh Aloe vera leaves to yield a slurry;
b) mixing 0.1 to 10 grams of each parent constituent into a separate portion of slurry that is approximately 10 to 1000 grams, waiting 24 hours after each addition;
c) mixing the separate slurry mixtures to form one slurry mixture of approximately 40 to 4000 grams;
d) passing the slurry mixture of c) through a separator to separate the liquids from the solids;
e) adding 1-50% by weight of honey to the liquid obtained from d);
f) mixing the components of e).

11. A method of using the composition of claim 1, comprising:
a) applying the composition to clean skin;
b) allowing the composition to dry on the skin;
c) retaining the dried composition on the skin for approximately 8 hours;
d) washing the composition from the skin;
e) repeating steps a) through d).

12. A method of using the composition of claim 2, comprising:
a) applying the composition to clean skin;
b) allowing the composition to dry on the skin;
c) retaining the dried composition on the skin for approximately 8 hours;
d) washing the composition from the skin;
e) repeating steps a) through d).

13. A method of using the composition of claim 3, comprising:
a) applying the composition to clean skin;
b) allowing the composition to dry on the skin;
c) retaining the dried composition on the skin for approximately 8 hours;
d) washing the composition from the skin;
e) repeating steps a) through d).

14. A method of using the composition of claim 4, comprising:
a) applying the composition to clean skin;
b) allowing the composition to dry on the skin;
c) retaining the dried composition on the skin for approximately 8 hours;
d) washing the composition from the skin;
e) repeating steps a) through d).

15. A method of using the composition of claim 5, comprising the steps of:
a) applying the composition to clean skin;
b) allowing the composition to dry on the skin;
c) retaining the dried composition on the skin for approximately 8 hours;
d) washing the composition from the skin;
e) repeating steps a) through d).

16. A method to re-grow hair, said method comprising the steps of:
applying the composition of claim 1 to a bald area;
b) allowing the composition to dry on the skin;
c) retaining the dried composition on the skin for approximately 8 hours;
d) washing the composition from the skin;
e) repeating steps a) through d) until hair re-growth is observed.

17. The composition of claim 1, wherein the composition is included in a shampoo.

18. The composition of claim 2, wherein the composition is included in a shampoo.

19. The composition of claim 3, wherein the composition is included in a shampoo.

20. The composition of claim 4, wherein the composition is included in a shampoo.

21. The composition of claim 5, wherein the composition is included in a shampoo.

22. The composition of claim 1, wherein the composition is included in a face wash.

23. The composition of claim 2, wherein the composition is included in a face wash.

24. The composition of claim 3, wherein the composition is included in a face wash.

25. The composition of claim 4, wherein the composition is included in a face wash.

26. The composition of claim 5, wherein the composition is included in a face wash.

27. The composition of claim 1, wherein the composition is included in a body wash.

28. The composition of claim 2, wherein the composition is included in a body wash.

29. The composition of claim 3, wherein the composition is included in a body wash.

30. The composition of claim 4, wherein the composition is included in a body wash.

31. The composition of claim 5, wherein the composition is included in a body wash.
